SEQUENCE LISTING (4 sequences)

SEQ ID NO: 1 - DNA, 20 bp, artificial sequence
  Forward primer of  GAPDH
ccgcatcttcttgtgcagtg

SEQ ID NO: 2 - DNA, 20 bp, artificial sequence
  Reverse primer of GAPDH
tcccgttgatgaccagcttc

SEQ ID NO: 3 - DNA, 20 bp, artificial sequence
  Forward primer of BDDhFVIII
gattctggggtgccacaact

SEQ ID NO: 4 - DNA, 21 bp, artificial sequence
  Reverse primer of BDDhFVIII
ctggtgggttttgagagaagc

(19) United States
(12) Patent Application Publication
(10) Pub. No.: US 2020/0370019 A1
(43) Pub. Date: Nov. 26, 2020

(54) RECOMBINANT ADIPOSE-DERIVED STEM CELL EXPRESSING BDDHFVIII GENE, AND PREPARATION METHOD AND APPLICATION THEREOF

(71) Applicant: North China University of Science and Technology, Hebei (CN)

(72) Inventors: Zhenyu YAN, Hebei (CN); Yanyan XIE, Hebei (CN); Linhong WANG, Hebei (CN); Yiwen ZHU, Hebei (CN)

(21) Appl. No.: 16/686,910

(22) Filed: Nov. 18, 2019

(30) Foreign Application Priority Data
May 20, 2019 (CN) 201910418282.X

(51) Int. Cl. C12N 5/0775; C12N 15/86; G01N 1/40; A61K 35/28; C07K 14/755

(52) U.S. Cl. CPC C12N 5/0667; C12N 15/86; C12N 2750/14141; A61K 35/28; C07K 14/755; G01N 1/4044

(57) ABSTRACT

The present invention provides a recombinant adipose-derived stem cell expressing a BDDhFVIII gene, and a preparation method and application thereof, and belongs to the technical field of genetically engineered drugs. The recombinant adipose-derived stem cell is obtained by infecting an adipose-derived stem cell with an adenoviral vector expressing the BDDhFVIII gene. The preparation method includes the following steps: constructing an adenoviral vector expressing a BDDhFVIII gene; extracting an adipose-derived stem cell; and infecting the adipose-derived cell with the adenoviral vector expressing the BDDhFVIII gene to obtain the recombinant adipose-derived stem cell. The recombinant adipose-derived stem cell can express the blood coagulation factor VIII safely and persistently, and have a high application prospect for treating the hemophilia A.

### CROSS REFERENCE TO RELATED APPLICATIONS AND DOCUMENTS

This application claims priority to Chinese application number 201910418282.X, filed May 20, 2019, entitled RECOMBINANT ADIPOSE-DERIVED STEM CELL EXPRESSING BDDHFVIII GENE, AND PREPARATION METHOD AND APPLICATION THEREOF, which is incorporated herein by reference in its entirety.

Further, this patent application incorporates by reference the Sequence Listing file enclosed herewith having the file name “SEQ.LISTING_ST25.txt” which is comprised of 1,164 bytes and has a date of creation of Jul. 10, 2019.

### TECHNICAL FIELD

The present invention belongs to the technical field of genetically engineered drugs, and in particular to a recombinant adipose-derived stem cell expressing a BDDhFVIII gene, and a preparation method and application thereof.

### BACKGROUND

Hemophilia A is the most common hereditary hemorrhagic disease in the clinic due to the defect or dysfunction of a gene that encodes a blood coagulation factor VIII (FVIII). A patient with severe hemophilia A has an activity level of the blood coagulation factor in the plasma thereof less than 1% (the normal range is 50%-150%), and is prone to suffer from uncontrolled bleeding after spontaneous and mild trauma, or severe bleeding after wounding or a surgery. Bleeding sites are mainly composed of deep muscles and weight-bearing joints. It is easy to form chronic pain and joint deformity if the sites are not cured thoroughly. If bleeding occurs in a central nervous system, the mortality rate would be extremely high. The incidence of the hemophilia A is about 1/10000, and since the population base of China is large, the number of patients suffering from the onset of hemophilia has exceeded 10,000 according to incomplete statistics, where the proportion of children and young and middle-aged patients is extremely high, and the hemophilia A is an important reason for the drop-out rate of child patients and the unemployment of young and middle-aged patients. Therefore, hemophilia is receiving increasing attention due to its serious threat to the life quality of hemophilia patients. In developed countries, the current treatment for the hemophilia A is the prophylactic intravenous infusion of a genetically engineered product, i.e., the blood coagulation factor VIII. Maintaining the activity level of FVIII in the plasma above 1% can effectively reduce the frequency of spontaneous bleeding, and maintaining the activity level of FVIII in the plasma above 5% can turn a severe type to a mild type. However, the recombinant blood coagulation factor, regardless of being extracted from the plasma or synthesized through genetic engineering, has a short half-life, needs frequent infusion, and has an expensive price, which imposes a great burden on the patient's family and the government. In developing countries, treatment is primarily an on-demand infusion. The biggest side effect of a replacement therapy in which the blood coagulation factor is infused repeatedly into the hemophilia patient for a long term is producing irreversible inhibitors, and the efficiency of the replacement therapy is significantly reduced after the formation of the inhibitors, thereby increasing the risk of bleeding again. Since the replacement therapy is a non-cure method that requires lifelong medication and brings a huge economic burden, more than 70% of the hemophilia patients worldwide cannot afford the huge cost of using the blood coagulation factor. Therefore, gene therapy is the only way that is hopeful to cure the hemophilia A. The hemophilia A, as an X-linked single-gene genetic hemorrhagic disease, accounts for 80%-85% of the total number of hemophilia. The current blood-coagulation-factor replacement therapy “cure the symptoms, not the disease”, and has limitations. The hemophilia A can only be completely cured by correcting the FVIII gene defect at a gene level. Reviewing domestic and foreign literatures, it is found that the gene therapy for hemophilia B is relatively mature and successfully applied in the clinic, while the gene therapy for the hemophilia A progresses slowly, and the selection of the viral vector and target cell thereof has not been determined. The hemophilia A has a higher incidence than the hemophilia B, and has serious bleeding in the clinic. It is urgent to improve the symptoms of the hemophilia A completely by gene therapy.

### SUMMARY

In view of this, an objective of the present invention is to provide a recombinant adipose-derived stem cell expressing a BDDhFVIII gene, and a preparation method and application thereof, the recombinant adipose-derived stem cell expressing the BDDhFVIII gene having an advantage of being capable of expressing a blood coagulation factor VIII safely and persistently.

In order to achieve the foregoing invention objective, the present invention provides the following technical solutions.

A recombinant adipose-derived stem cell expressing a BDDhFVIII gene is disclosed, which is obtained by infecting an adipose-derived stem cell with an adenoviral vector expressing the BDDhFVIII gene.

Preferably, the adipose-derived stem cell is derived from an inguinal adipose tissue.

Preferably, the adenoviral vector is an adenoviral vector of AD5 type.

The present invention provides a method for preparing the recombinant adipose-derived stem cell, including the following steps:

constructing an adenoviral vector expressing a BDDhFVIII gene;

extracting an adipose-derived stem cell;

infecting the adipose-derived stem cell with the adenoviral vector expressing the BDDhFVIII gene to obtain the recombinant adipose-derived stem cell.

Preferably, the extracting of the adipose-derived stem cell is conducted by using a collagenase digestion method.

Preferably, the adipose-derived stem cell is extracted from the inguinal adipose tissues on both sides of an SD rat.

Preferably, the temperature for the collagenase digestion is 36-38° C.; and the time for the collagenase digestion is 1-1.5 h.

Preferably, after extracting the adipose-derived stem cell, the method further includes identifying the obtained adipose-derived stem cell.

Preferably, the infection has a multiplicity of infection value of 300-400.

The present invention also provides the use of the recombinant adipose-derived stem cells in preparing a medicament for preventing and treating hemophilia A.

Beneficial Effects of the Present Invention:

the recombinant adipose-derived stem cell expressing the BDDhFVIII gene as provided by the present invention is obtained by infecting the adipose-derived cell with the adenoviral vector expressing the BDDhFVIII gene. The recombinant adipose-derived stem cell can express the blood coagulation factor VIII safely, persistently and effectively, and have a good application prospect for treating the hemophilia A.

### BRIEF DESCRIPTION OF THE DRAWINGS

FIGS. 1A, 1B and 1C show the morphology of ADSCs under an inverted microscope (×100), namely, FIG. 1A: subjected to primary culture for 48 h; FIG. 1B: subjected to primary culture for 7 d; and FIG. 1C: the ADSCs of the 3rd generation;

FIG. 2 shows the growth curve of the ADSCs of the 3rd generation;

FIGS. 3A, 3B and 3C show the induction and identification of ADSCs under the inverted microscope (×100), wherein FIG. 3A: ALP staining 7 days after osteogenic induction; FIG. 3B: oil red O staining 14 days after adipogenic induction; and FIG. 3C: alizarin red staining 30 days after osteogenic induction;

FIG. 4 shows the flow-cytometry detection results of a surface marker of the cells of the 3rd generation;

FIGS. 5A, 5B, 5C, 5D, 5E and 5F show the cell morphology of infected ADSCs observed under the inverted microscope (×100), wherein FIG. 5A: normal ADSCs after 24 h; FIG. 5B: ADSCs infected with Ad-BDDhFVIII-GFP for 24 h; FIG. 5C: normal ADSCs after 48 h; FIG. 5D: ADSCs infected with Ad-BDDhFVIII-GFP for 48 h; FIG. 5E: normal ADSCs after 72 h; and FIG. 5F: ADSCs infected with Ad-BDDhFVIII-GFP for 72 h;

FIG. 6 shows the effect of infection on the proliferation of ADSCs in the three groups of Embodiment 1;

FIG. 7 shows the hFVIIIAg level 72 hours after the ADSCs are infected in Embodiment 1;

FIG. 8 shows the expression of the BDDhFVIII gene after the ADSCs are infected in Embodiment 1; and

FIG. 9 shows the level of an hFVIII protein 72 hours after the ADSCs are infected.

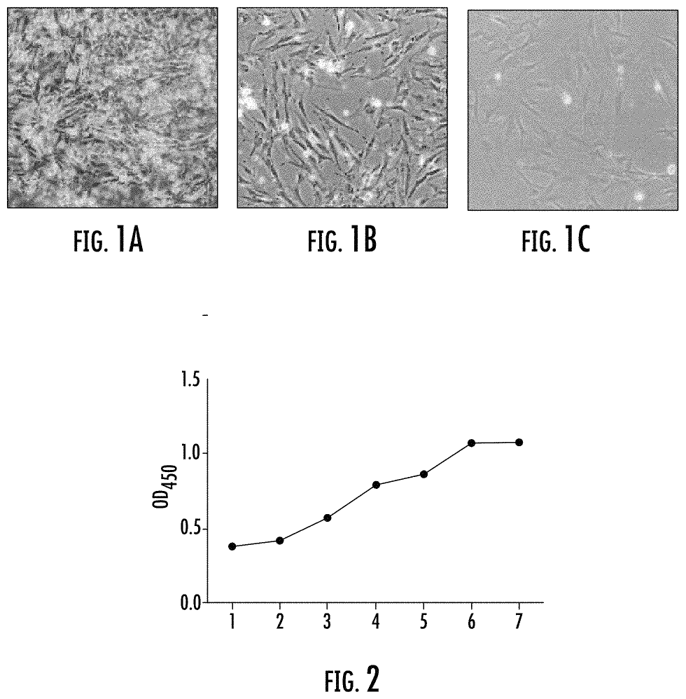

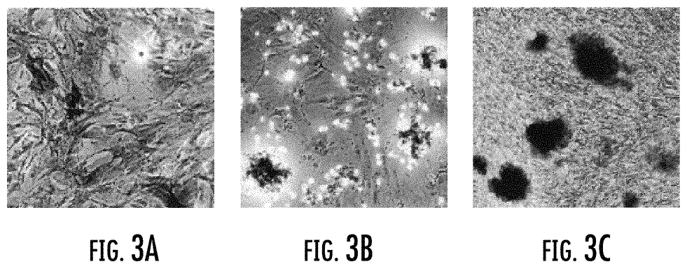

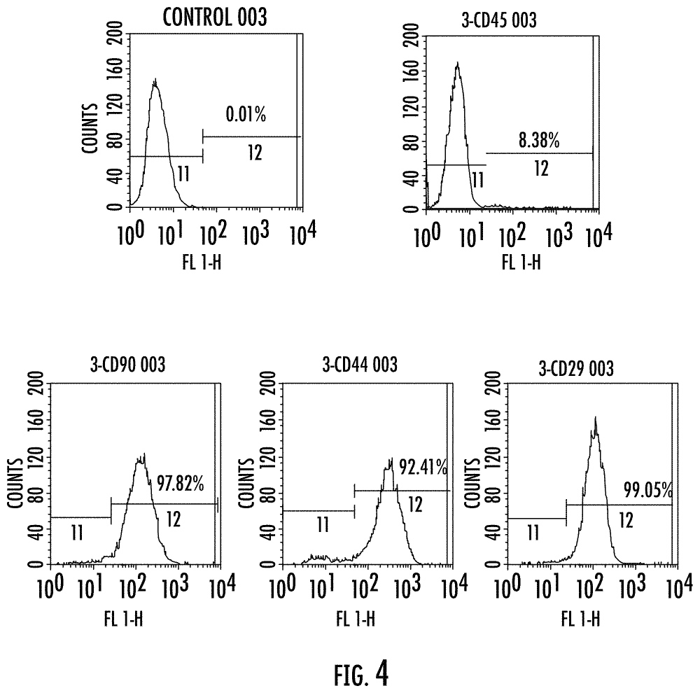

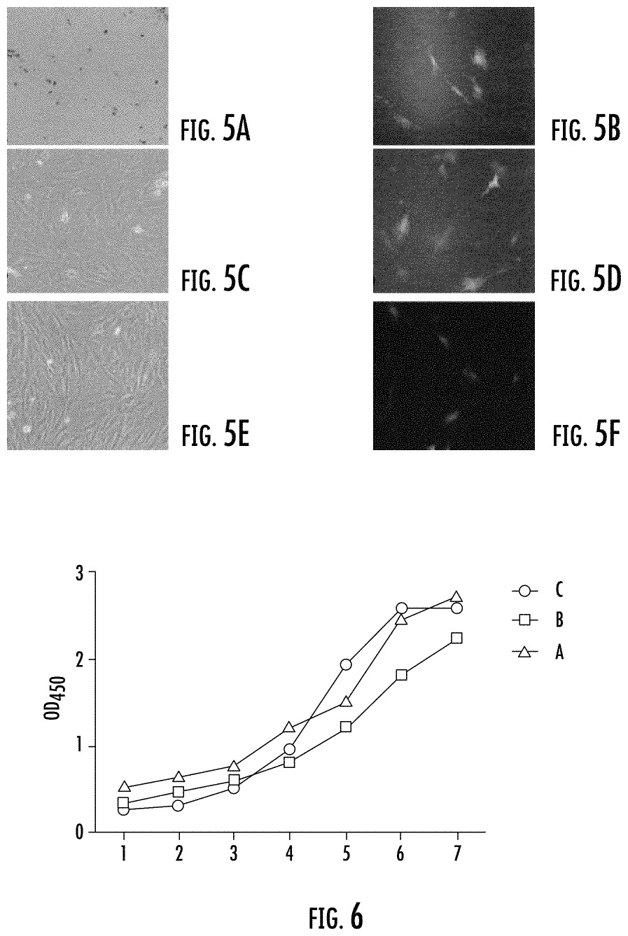

### DETAILED DESCRIPTION

The present invention provides a recombinant adipose-derived stem cell expressing a BDDhFVIII gene, which is obtained by infecting an adipose-derived stem cell with an adenoviral vector expressing the BDDhFVIII gene.

In the present invention, the adipose-derived stem cells are preferably derived from an SD rat, and more preferably from an inguinal adipose tissue of the SD rat. In the present invention, the adenoviral vector is preferably an adenoviral vector of the AD5 type; and the adenoviral vector is preferably an adenoviral vector that expresses a green fluorescent protein. The present invention has no specific limitation on the source of the adenoviral vector, and both an adenoviral vector obtained by synthesizing through a conventional method in the art or a commercially-available adenoviral vector can be used.

The present invention provides a method for preparing the recombinant adipose-derived stem cell, including the following steps: constructing an adenoviral vector expressing a BDDhFVIII gene; extracting an adipose-derived stem cell; and infecting the adipose-derived cell with the adenoviral vector expressing the BDDhFVIII gene to obtain the recombinant adipose-derived stem cell.

In the present invention, constructing the adenoviral vector expressing the BDDhFVIII gene preferably includes packaging the BDDhFVIII gene with an adenoviral vector to obtain the adenoviral vector expressing the BDDhFVIII gene. In the present invention, the BDDhFVIII gene is a gene of the human blood coagulation factor VIII having the deletion of a B region, and is a sequence after 2657-5141 is deleted from the gene FVIII; and the activity and function of the BDDhFVIII gene are both consistent with those of the gene of the human blood coagulation factor VIII having no deletion of the B region. In the present invention, the method for constructing the adenoviral vector expressing the BDDhFVIII gene is referenced in a HB-infusion seamless cloning kit of Hanbio Biotechnology Co., Ltd. (Shanghai); and in the specific implementation process of the present invention, preferably the adenoviral vector is constructed by the Hanbio Biotechnology Co., Ltd. (Shanghai).

In the present invention, extracting the adipose-derived stem cell is preferably conducted by a collagenase digestion method; and in the present invention, the adipose-derived stem cell is preferably extracted from the inguinal adipose tissues on both sides of an SD rat. During the specific implementation of the present invention, the method includes the following steps: S1) separating the inguinal adipose tissues on both sides after the SD rat is anesthetized intraperitoneally and disinfected; S2) washing and chopping the adipose tissues, and performing collagenase digestion to obtain digested cells; S3) screen-filtering and centrifuging the digested cells to collect a solid-phase component as the adipose-derived stem cell.

In the present invention, the inguinal adipose tissues on both sides are separated after the SD rat is anesthetized intraperitoneally and disinfected. The reason for selecting the inguinal adipose tissues in the present invention is that the inguinal adipose tissues are easily obtained and has a large yield, and the source thereof is sufficient; while the fat in other places is of a small amount, difficult to acquire or easy to be polluted during extraction. The present invention has no special requirements on the method of intraperitoneal anesthesia, and an SD-rat intraperitoneal anesthesia method which is conventional in the art can be used. In the present invention, the disinfection is preferably performed by alcohol sterilization, which is specifically soaking the SD rat in alcohol for 5-10 min; and the alcohol is preferably 75% by volume of alcohol. In the present invention, after the disinfection, the inguinal adipose tissues on both sides are separated by a tweezer.

In the present invention, after the adipose tissues are obtained by separating, the adipose tissues are washed and chopped, and then subjected to collagenase digestion to obtain the digested cells. In the present invention, the washing solution for washing is preferably a PBS solution containing a bispecific antibody at a volume fraction of 1% (the bispecific antibody is specific to both penicillin and streptomycin), the number of washing is preferably 3-4 times, and the washing is preferably carried out in a culture dish; and in the specific implementation of the present invention, the washing washes off blood vessels and residual substances from the adipose tissues as much as possible. In the present invention, after the washing is completed, the washed adipose tissue is chopped. In the present invention, the washed adipose tissues are transferred into a new container, and the adipose tissues are chopped; the present invention has no specific requirement on the chopping method, and a tissue chopping method conventional in the art can be used. In the specific implementation of the present invention, a method of chopping manually by means of a scissor is used, and the time for the chopping is preferably 30-40 min; and in the present invention, preferably the adipose tissues are chopped until a paste is formed. In the present invention, the collagenase digestion is conducted after the chopping; a collagenase solution is mixed with the chopped adipose tissues for collagenase digestion; the volume ratio of the collagenase solution to the chopped adipose tissues is preferably (1.5-2.5):1, and more preferably 2:1; and the concentration of the collagenase solution is preferably 0.5%-1.5% (v/v), and more preferably 1% (v/v). In the present invention, the collagenase is preferably a collagenase of type I. In the present invention, the temperature for the collagenase digestion is preferably 36-38° C., and more preferably 37° C.; and the time for the collagenase digestion is preferably 1-1.5 h, and more preferably 1.1-1.4 h. In the present invention, during the digesting process preferably manual shaking is conducted for 3-4 times, and the time of each manual shaking is preferably 10-15 min. In the present invention, after the end of the collagenase digestion, the termination of the digestion is preferably carried out. In the present invention, the termination of the digestion is preferably adding an equal volume of a DMEM/F12 complete medium containing 10% FBS into the digested tissues.

In the present invention, the digested cells are screen-filtered, and centrifuged to collect solid-phase components as the adipose-derived stem cells. In the present invention, the pore size for the screen filtering is preferably 200 mesh; the centrifugal rotation speed is preferably 800-1,200 rpm, and more preferably 1,000 rpm; and the time for centrifugation is preferably 8-12 min, and more preferably 10 min. In the present invention, after the centrifugation, the solid-phase components are collected to obtain adipose-derived stem cells; the obtained adipose-derived stem cells are preferably cultured by resuspending in a complete medium; and the culture is preferably inoculating into a culture flask, and placing into an incubator containing 5% CO₂ for conducting culture at 37° C. In the present invention, after extracting the adipose-derived stem cell, the method further includes identifying the obtained adipose-derived stem cell. In the present invention, the purpose of the identification is to determine whether the extracted adipose-derived stem cell has the characteristics of a stem cell; the identification preferably includes the identification of osteogenesis and adipogenesis; and the present invention has no specific limitation on the identification method, and a method conventional in the art can be used.

In the present invention, after the adipose-derived stem cell is obtained, the adipose-derived stem cell is infected with the adenoviral vector expressing the BDDhFVIII gene to obtain the recombinant adipose-derived stem cell. In the present invention, the adipose-derived stem cell is preferably an adipose-derived stem cell sub-cultured to a third generation; and the concentration of the adipose-derived stem cell of the third generation is preferably (0.1-5)×10⁵ cells/ml, more preferably 0.5-2×10⁵ cells/ml, and most preferably 1×10⁵ cells/ml. In the specific implementation of the present invention, the adipose-derived stem cell at the concentration is preferably inoculated on a cell culture plate for culture, and the virus infection is conducted when the cells are grown to 70-80%. In the present invention, the multiplicity of infection (MOI) value of the virus infection is preferably 300-400, and more preferably 350. In the present invention, the adenoviral vector expressing the BDDhFVIII gene is added in an amount of preferably (1-5)×10¹⁰ pfu/mL, and more preferably 1.26×10¹⁰ pfu/mL; the method for calculating the added volume of virus in the present invention is: MOI=viral amount (ml)×virus titer (pfu/mL)/number of cells (cells). In the present invention, after the cells are infected for 15-20 min, the cell culture plate is preferably shaken, such that the adenoviral vector expressing the BDDhFVIII gene is sufficiently contacted with the adipose-derived stem cell, and a complete medium is replaced 24 h after the infection of the cells for culturing continually. In the present invention, preferably the fluorescent expression of the cells is observed under a fluorescence microscope at 24 h, 48 h, and 72 h after the infection of the cells, to determine that the BDDhFVIII carried by the adenovirus can successfully infect the adipose-derived stem cell.

The present invention also provides the use of the recombinant adipose-derived stem cells in preparing a medicament for preventing and treating hemophilia A. In the present invention, the recombinant adipose-derived stem cell is capable of expressing the gene of the human blood coagulation factor VIII.

The technical solution provided by the present invention will be described in detail in connection with the following embodiments, but they should not be construed as limiting the claimed scope of the present invention.

### Embodiment 1

Extraction of Adipose-Derived Stem Cell

2 commercially-available SD rats, whether of male or female, were taken, and weighed about 120 g. The SD rats were subjected to intraperitoneal anesthesia. The rats were immersed in 75% alcohol from the neck thereof down for 10 min for disinfection. After disinfection, the SD rats were transferred into a biosafety cabinet; the inguinal adipose tissues on both sides of the SD rats were carefully separated by a tweezer, and the separated fat was placed into and washed in a culture dish containing 1% of a bispecific antibody in PBS for 3-4 times, to wash off the blood vessels and residual substances from the adipose tissues as much as possible, and then the adipose tissues were transferred into a small beaker and chopped thoroughly (chopped until a paste is formed) for about 35 min. The chopped adipose tissues were transferred into a 50 ml centrifuge tube and added with 2 volumes of 0.1% (v/v) type I collagenase, the centrifuge tube was sealed with a sealing membrane and placed into a 37° C. water bath tank to conducting digestion for 1.3 h, and during the digestion the tube was appropriately taken out and shaken manually for 4 times, each time lasting 13 min After the adipose tissues were completely digested (as long as no massive microstructure was observed), digestion was terminated by adding an equal amount of a DMEM/F12 complete medium containing 10% FBS. The cells were filtered through a screen of 200 mesh, centrifuged at 1,000 rpm for 10 min, resuspended in 5 ml of a complete medium, inoculated into a 25 cm² culture flask, and cultured in an incubator containing 5% CO₂ at 37° C.

Recombinant Adenoviral Vector

The carried gene is the gene of the human blood coagulation factor VIII having a deletion in the region B (BDDFVIII), (the sequence of 2657-5141 in the gene FVIII is the B region); the adenovirus was purchased from the Hanbio Biotechnology Co., Ltd. (Shanghai) and of type AD5, and the recombinant adenoviral vector Ad-BDDhFVIII-GFP was constructed by the Hanbio Biotechnology Co., Ltd. (Shanghai).

Infecting Adipose Tissue Stem Cells with a Recombinant Adenoviral Vector

Adipose tissue stem cells of the P3 generation were taken to prepare a single-cell suspension at the adjusted density of 1×10⁵/ml, and then inoculated onto a 6-well plate. The multiplicity of infection (MOI) value of 350 was taken when the cells grew to 70-80%. A recombinant adenoviral vector Ad-BDDhFVIII-GFP (1.26×10¹⁰ pfu/mL) was added into the cells. The method for calculating the added volume of virus was: MOI=viral amount (ml)×virus titer (pfu/mL)/number of cells (cells). 15-20 min after the infection of the cells, the 6-well plate was gently shaken to allow the virus solution to fully contact with the cells, and a complete medium was replaced after 24 h to continue the culture. The fluorescent expression of the cells was observed under a fluorescence microscope at 24 h, 48 h, and 72 h after the infection of the cells with the virus.

The proliferation conditions of the transfected cells were detected by using a CCK-8 method, as shown FIG. 6, and the results showed that: all of three groups of cells entered the logarithmic growth phase when cultured for 3 d, the growth of the cells entered the plateau phase after 6 days, and all of the three groups of cells were grown with an inverted “S” growing curve; and the comparison of the OD values among the three groups within 7 days showed a difference of statistical significance (P<0.05), indicating that for the groups A and B, the infection had an effect on the cell proliferation.

Among them: Group A: a recombinant adenoviral vector Ad-BDDhFVIII-GFP that carrying the BDDhFVIII gene and expressing GFP was infected into the ADSCs of SD rats to observe the expression conditions of the BDDhFVIII in the ADSCs.

Group B: an adenoviral vector Ad-GFP only expressing the GFP was infected into the ADSCs of the SD rats to observe the expression conditions of the BDDhFVIII in the ADSCs.

Group C: normally-cultured ADSCs without any treatment.

Detection of the BDDhFVIII Gene in Infected Cells of Each Group by RT-PCR

Steps for RT-PCR

The total RNA of the three groups was extracted by a TRIZOL method.

The specific operations of extracting RNA was as follows

1. The culture flask was rinsed with a PBS solution for 2-3 times, the liquid in the culture flask was aspirated, and the culture flask was added with 1 mL of TRIZOL, placed on ice for 20 min and meanwhile shaken to make the liquid fully contact with the cells, the cells were pipetted up and down to make the cells fall off and mix well with the TRIZOL liquid, and the mixture was transferred into a 1.5 ml enzyme-free EP tube.

2. The EP tube was added with 200 μL of chloroform, manually turned upside down for 15 s for mixing well, and let to stand at room temperature for 3-5 min.

3. The cells were centrifuged at 12,000 rpm at 4° C. for 15 min.

4. After centrifugation, the liquid in the EP tube was divided into 3 layers, with the uppermost colorless aqueous sample being RNA, and 400-500 μL of the uppermost colorless aqueous sample was transferred to a new enzyme-free EP tube (it should be noted that the middle layer should not be pipetted to avoid RNA degradation or pollution).

5. The new enzyme-free EP tube was added with an equal volume of pre-cooled isopropanol (with the amount being greater than or equal to the pipetted amount), turned upside down for 3 times for mixing well, and let to stand at room temperature for 10 min.

6. The tube was centrifuged at 12,000 rpm at 4° C. for 15 min, and meanwhile 1 mL of 75% cold ethanol was prepared and placed in a refrigerator at 4° C. for pre-cooling (750 μl of absolute ethanol+250 μl of DEPC).

7. The tube was let to stand at room temperature for 5 min after the centrifugation, such that the RNA was completely precipitated, the supernatant was discarded, the tube was added with 1 mL of the 75% cold ethanol and turned upside down for several times, and the RNA precipitate was washed.

8. The supernatant was discarded, the precipitate was dried at room temperature for about 8 min (ensuring that no residual ethanol was remained in the tube, and avoiding excessive drying of the RNA), and the RNA was dissolved in 10 μL of enzyme-free DEPC-treated water.

9. 1 μL of each of the RNA samples was pipetted using a micropipette, and the RNA concentration and the OD value at 260/280 nm were determined by a Q5000 software, where the A260/280 in the range between 1.8-2.0 was regarded as reaching the standard, and the total RNA concentration of the cells was measured on a machine. After measuring of the RNA concentration, the RNA was reverse-transcribed into cDNA according to a Takara reverse transcription kit (Takara, USA, Cat. No.: RR037A). By using rat GAPDH (250 bp) as an internal reference, a pair of primers specific to the gene sequence of the human blood coagulation factor VIII (179 bp) that span the domain of the B region were designed (Table 1), the primer synthesis was performed by Shanghai Invitrogen Co., Ltd., and the subsequent PCR amplification of the specific sequences was specifically operated as follows.

TABLE 1
Primer Sequence

| | Forward primer | Reverse primer |
|---|---|---|
| Gene | (5′ to 3′) | (5′ to 3′) |
| GAPDH | CCGCATCTTCT | TCCCGTTGATG |
| | TGTGCAGTG | ACCAGCTTC |
| | (SEQ ID NO: 1) | (SEQ ID NO: 2) |
| BDDhFVIII | GATTCTGGGGT | CTGGTGGGTTT |
| | GCCACAACT | TGAGAGAAGC |
| | (SEQ ID NO: 3) | (SEQ ID NO: 4) |

1. Dilution of the upstream primer: an EP tube containing the upstream primer was centrifuged at 4,000 rpm for 4-5 min, added with 78 μL of DEPC water after the cap of the tube was slowly opened, and then shaken well after the cap of the tube was closed. 5 μL was taken and poured into another EP tube, and the another EP tube was added with 45 μL of DEPC water for being ready for use.

2. Dilution of the downstream primer: an EP tube containing the downstream primer was centrifuged at 4,000 rpm for 4-5 min, added with 98 μL of DEPC water after the cap of the tube was slowly opened, and then shaken well after the cap of the tube was closed. 5 μL was taken and poured into another EP tube, and the another EP tube was added with 45 μL of DEPC water for being ready for use.

3. For the three groups, PCR was performed using a cRNA as a template, and 25 μL of a total reaction system was constructed, with three replicate wells for each group.

4. 25 μL of a reaction system: 2 μL of cDNA, 1 μL of PrimeOne, 1 μL of PrimerTwo, 12.5 μL of 2×MasterMix, and ddH₂O being added to 25 μL.

5. Reaction conditions: 94° C. for 3 min, 94° C. for 30 s, 60° C. for 45 s, 72° C. for 1 min, 72° C. for 5 min, for 30 cycles. The product was identified by agarose gel electrophoresis.

The specific operations of the agarose gel electrophoresis were as follows

1. Formulation of 1% agarose gel: 0.25 g of agarose powder was weighed and dissolved in 25 ml of a mixed solution (20 mL of ultra-pure water+5 mL of a TBE solution), and placed into a triangular flask for being ready for use. 2. The formulated buffer was poured into the triangular flask and heated in a microwave oven for dissolving, the triangular flask was carefully shaken when the solution boiled, so as to completely and uniformly dissolve the agarose, and this step was repeated for several times until the agarose was completely dissolved. 3. When the solution was cooled to 60° C., 4 μL EB substitute was added and mixed well. The agarose solution was poured into a gel-making die, then the comb was inserted at appropriate positions, the gel thickness was set between 3-5 mm, and the gel was solidified at 4° C. for about 60 min. 4. About 5 μL of the sample was taken and added sequentially into the sample tanks. 5. After the sample was added, the electrophoresis tank cover was closed and the power was immediately turned on, the starting voltage was kept at 90 v, and when the band moved to the distance of about 2 cm away from the front edge of the gel, the electrophoresis was stopped, the images were observed and photographs thereof were taken for preservation.

The detection results were as shown in FIG. 8, and the results showed that: the expression of the internal reference gene was observed 72 hours after infection of the ADSCs in each of the three groups. The group A had the results that were consistent with the length of the amplified fragment and had the expression of the BDDhFVIII gene; and no target band was seen and thus no BDDhFVIII gene was expressed in each of groups B and C.

Detection of the Expression Level of the FVIII Antigen in the Cell Supernatant by ELISA

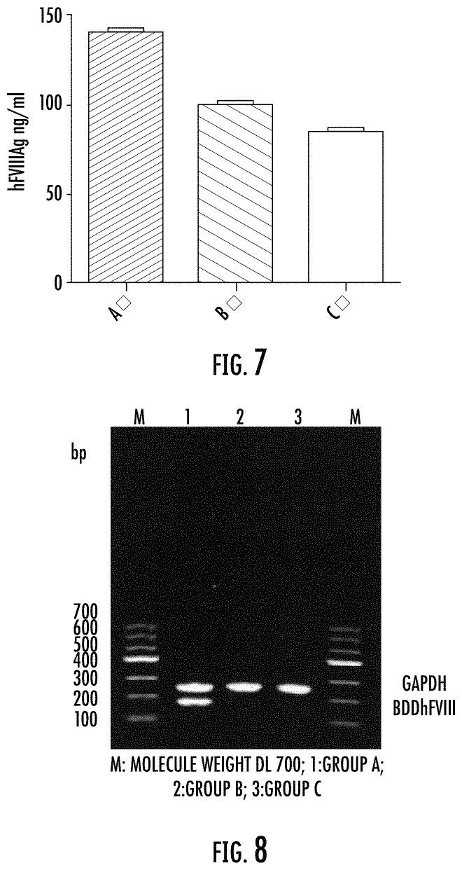

The ELISA kit was purchased from BeiJing Andygene Biotechnology Co., Ltd, where the specific method was referred in the instructions of the kit, the detection results were shown in FIG. 7, and the levels of hFVIIIAg in the supernatants of infected cells in the three groups of A, B and C showed that: hFVIIIAg expression could be detected in the cell supernatant within 72 h after the infection of the ADSCs in each of the three groups, where the group A had the maximum hFVIIIAg expression, and the group C had the minimum hFVIIIAg expression, the differences in hFVIIIAg expression among the three groups were statistically significant (P<0.001)

Expression Conditions of the BDDhFVIII Gene in Cells of Each Group after Infection as Detected by Western Blot

Cells of the P3 generation were taken for experiments, the cells were infected according to the three groups of A, B and C, the total protein was extracted 72 h after the infection was successful.

The protein extraction process was as follows (the whole process was performed on ice)

1. The infected cells were taken out from the incubator, and the medium was discarded. 2. Preformulation of protein lysate: a RIPA lysis buffer:1% of PMSF=1:100. 3. Each culture flask was added with 2-3 mL of a PBS solution, and gently shaken to rinse the cells for 3-4 times, and for the last time of rinsing, the residual PBS was aspirated with a 1 mL pipette to prevent the lysate from being diluted to affect the effect. 4. The cell culture flask was added with the formulated lysis buffer at the amount of 120 μL, and the culture flask was placed flat on ice to enable the cells to be sufficiently lysed for 40 min. 5. The lysed cells were scraped to the bottom of the flask by a cell scraper, and the liquid was aspirated into a 1.5 ml EP tube using a 1 mL pipette. 6. The solution was centrifuged at 12000 rpm under 4° C. for 15 min. 7. The supernatant was transferred to a new 1.5EP tube after centrifugation, and (protein sample:5×loading buffer=4:1) was added in proportion after quantification of the BCA protein, mixed well, and then subjected to thermal denaturation in a metal bath at 100° C. for 10 min, and the denatured protein samples were cooled and transferred to −80° C. for storage.

(2) Preparing for gel making (the process was as follows and was conducted at normal temperature)

1. The glass plate was washed with clean water, rinsed with fine flowing water, then rinsed with ultrapure water, and air dried on a support, and a separation gel was formulated according to the gel formulation instructions. 2. The long and short plates of the glass plates were aligned (the short plate faced inward), then clamped in a electrophoresis clamp, and placed on a gel maker for clamping, the separation gel solution was extracted using a 10 mL syringe and infused into a gap between the long and short plates along the glass plates, the infusion was stopped when the level of the gel rises to 1.5 cm from the top end, and the other side is infused in the same manner. 1 mL of absolute ethanol was pipetted using a 1 mL pipette and added from the top end of the separation gel for pressing the gel. After 30-40 min, a bright fold line appeared between the absolute ethanol and the separation gel, indicating that the separation gel had been fully solidified. At this time, a spacer gel is formulated according to the gel formulation instructions, the absolute ethanol was drained along the plate wall, and the moisture was aspirated to dry with a filter paper. The spacer gel was infused in the same manner to fill the remaining space full, and a comb with 10 teeth was inserted to avoid the appearance of bubbles. After 20-30 min, the spacer gel was completely solidified and ready for use. 3. The inner chamber was filled full of a freshly prepared 1× electrophoresis solution, the comb was gently pulled out by squeezing both sides of the tooth comb with two hands and put into the electrophoresis tank, and the 1× electrophoresis solution was added into the outer chamber until the resistance wire was immersed, and the protein Marker and samples were added sequentially using the 10 μL pipette. 4. The electrophoresis device was connected to a power supply, firstly, the electrophoresis was conducted at a constant voltage of 90 v until the bromophenol blue entered the separation gel and a red Marker band appeared, and then the electrophoresis was conducted at an adjusted voltage of 120 V and was stopped when the band reached the bottom of the separation gel, and then the electrophoresis device was disconnected from the power supply. 5 A PVDF membrane was pre-cut (the PVDF membrane was immersed in methanol for 1 min for activation), and then placed in a tray containing a electroporation solution in the order of black support-sponge-filter paper-gel-PVDF membrane-filter paper-sponge-red support, a clamp was placed in the electroporation tank, the electroporation tank was filled full of the freshly formulated electroporation solution, put in a basin of an ice-water mixture for cooling, and connected to a power supply with attention to the positive and negative poles, the switch was switched on to conduct electroporation at a constant current of 220 mA for about 2 h, then the electroporation was stopped, and the power supply was disconnected. 6. The PVDF membrane was taken out and placed into an incubation box of 5% skim milk powder for incubating for 1.5-2 h. 7. After the membrane was blocked, the band was taken out, rinsed in TBST for once, placed into a TBST-diluted protein-specific antibody Factor VIII (1:1000), and incubated at 4° C. overnight (for primary antibody, the film was placed facing down to make the antibody in sufficient contact with the membrane), and on the next day, the PVDF membrane was carefully taken out with a tweezer and placed into an incubation box added with TBST to rinse for 4 times, with each time of rinsing being 10 min 8. The PVDF membrane was placed into an incubation box of TBST-diluted and HRP-labeled goat anti-rat IgG secondary antibody (1:5000) and incubated at room temperature on the shaker for 2 h, and after the end of the incubation, the PVDF membrane was placed into TBST incubation cartridge was incubation box added with TBST to rinse for 4 times, with each time of rinsing being 10 min, and after the membrane rinsing was completed, the PVDF membrane was subjected to coloring. 9. The PVDF membrane was spin-dried by spinning to remove the TBST thereon, placed on a coloring tray (attention should be payed to the positive and negative faces of the placed membrane), a formulated ECL coloring solution (60 L/band) was dropped onto the tiled PVDF membrane, the tray was put into a gel imager for automatically exposing and developing, and the images were preserved.

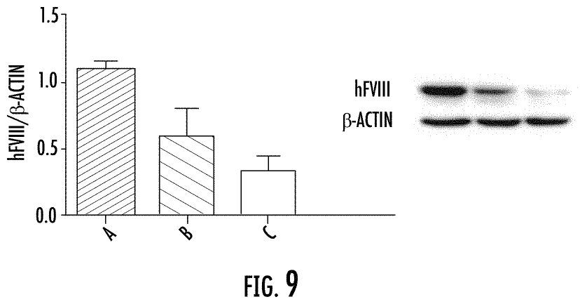

The Western blot results were as shown in FIG. 9, and the results showed that: expression of a hFVIIIAg protein could be detected in the cell supernatant within 72 h after the infection of the ADSCs in each of the three groups, where the group A had the maximum hFVIIIAg expression, and the group C had the minimum hFVIIIAg expression, the differences in the expression of the hFVIIIAg protein in the two groups of B and C as compared with the group A were statistically significant (P<0.001)

Animal Experiment of Factor-VIII-Gene Knockout SD Rats The factor-VIII-gene knockout SD rats (hemophilia A rats) (purchased from Shanghai Bioray Laboratories Inc.) were randomly numbered as 1-9, and 9 cards of the same size and same material were taken and numbered as 1-9. The cards corresponded to the rats, i.e. the card No. 1 corresponded to the rat No. 1, and so on. A lottery method was used to divide the rats into 3 groups of 3 each. The 3 groups were groups 1, 2 and 3 of hemophilia A rats, respectively. Normal SD rats were used as the fourth group.

Viral Vector and Target-Cell Injection

The viral vector was directly infused into the group A1 of hemophilia rats through the tail vein; a target cell containing the vector carrying the gene of interest, i.e., the recombinant adipose-derived stem cell infected with the adenoviral vector carrying the gene of the human blood coagulation factor VIII having the deletion of the B region, was infused into the group A2 of hemophilia rats through the tail vein; the adipose-derived stem cell transfected with the empty virus not carrying the gene of interest was infused into the group A3 of hemophilia rats through the tail vein; and the PBS buffer was infused into normal rats through the tail vein.

TABLE 2
Groups and injection dose

| | | Number of | Infusion | |
|---|---|---|---|---|
| Groups | Types of vectors | cells/virus titer | pathways | Dose |
| Group A1 of | Transfection of ADSCs with an | 1 × 10⁶/flask × | Tail vein | 100 μL/100 g |
| hemophilia rats | adenoviral vector carrying the FVIII | 2 flasks | injection | (body weight of |
| | gene | | | the rat) |
| Group A2 of | Transfection of ADSCs with an empty | 1 × 10⁶/flask × | Tail vein | 100 μL/100 g |
| hemophilia rats | adenoviral vector not carrying the | 2 flasks | injection | (body weight of |
| | FVIII gene | | | the rat) |
| Group A3 of | The group directly injected with the | 1.26 × 10¹⁰ | Tail vein | 300 μL/rat |
| hemophilia rats | adenoviral vector carrying the FVIII | pfu/ml | injection | |
| | gene | | | |
| Group A4 of | The group directly injected with the | 1.26 × 10¹¹ | Tail vein | 200 μL/rat |
| hemophilia rats | empty adenoviral vector not carrying | pfu/ml | injection | |
| | the FVIII gene | | | |
| Group A5 of | PBS | | Tail vein | 200 μL/rat |
| normal SD rats | | | injection | |

Venous Blood Sampling

Blood was sampled from the tail vein 1, 2, 7, 14, 21 and 28 days after the infusion of the viral vector and the target cell for functional and immunological detection, including partial thromboplastin time (APTT), factor VIII enzyme-linked immunosorbent assay (ELISA), anti-VIII factor antibody analysis, etc.; the aforementioned detection was entrusted to a detection mechanism.

The foregoing descriptions are only preferred implementation manners of the present invention. It should be noted that for a person of ordinary skill in the art, several improvements and modifications may further be made without departing from the principle of the present invention. These improvements and modifications should also be deemed as falling within the protection scope of the present invention.

What is claimed is:

1. A recombinant adipose-derived stem cell expressing a BDDhFVIII gene, wherein the recombinant adipose-derived stem cell is obtained by infecting an adipose-derived stem cell with an adenoviral vector expressing the BDDhFVIII gene.

2. The recombinant adipose-derived stem cell according to claim 1, wherein the adipose-derived stem cell is derived from an inguinal adipose tissue.

3. The recombinant adipose-derived stem cell according to claim 1, wherein the adenoviral vector is an adenoviral vector of AD5 type.

4. The recombinant adipose-derived stem cell according to claim 2, wherein the adenoviral vector is an adenoviral vector of AD5 type.

5. The recombinant adipose-derived stem cell according to claim 1, comprising the following steps:
constructing an adenoviral vector expressing a BDDhFVIII gene;
extracting an adipose-derived stem cell;
infecting the adipose-derived stem cell with the adenoviral vector expressing the BDDhFVIII gene to obtain the recombinant adipose-derived stem cell.

6. The recombinant adipose-derived stem cell according to claim 2, comprising the following steps:
constructing an adenoviral vector expressing a BDDhFVIII gene;
extracting an adipose-derived stem cell;
infecting the adipose-derived stem cell with the adenoviral vector expressing the BDDhFVIII gene to obtain the recombinant adipose-derived stem cell.

7. The recombinant adipose-derived stem cell according to claim 3, comprising the following steps:
constructing an adenoviral vector expressing a BDDhFVIII gene;
extracting an adipose-derived stem cell;
infecting the adipose-derived stem cell with the adenoviral vector expressing the BDDhFVIII gene to obtain the recombinant adipose-derived stem cell.

8. The recombinant adipose-derived stem cell according to claim 4, comprising the following steps:
constructing an adenoviral vector expressing a BDDhFVIII gene;
extracting an adipose-derived stem cell;
infecting the adipose-derived stem cell with the adenoviral vector expressing the BDDhFVIII gene to obtain the recombinant adipose-derived stem cell.

9. The recombinant adipose-derived stem cell according to claim 5, wherein the extracting of the adipose-derived stem cell is conducted by using a collagenase digestion method.

10. The recombinant adipose-derived stem cell according to claim 6, wherein the extracting of the adipose-derived stem cell is conducted by using a collagenase digestion method.

11. The recombinant adipose-derived stem cell according to claim 7, wherein the extracting of the adipose-derived stem cell is conducted by using a collagenase digestion method.

12. The recombinant adipose-derived stem cell according to claim 8, wherein the extracting of the adipose-derived stem cell is conducted by using a collagenase digestion method.

13. The recombinant adipose-derived stem cell according to claim 9, wherein the adipose-derived stem cell is extracted from the inguinal adipose tissues on both sides of an SD rat.

14. The recombinant adipose-derived stem cell according to claim 10, wherein the adipose-derived stem cell is extracted from the inguinal adipose tissues on both sides of an SD rat.

15. The recombinant adipose-derived stem cell according to claim 11, wherein the adipose-derived stem cell is extracted from the inguinal adipose tissues on both sides of an SD rat.

16. The recombinant adipose-derived stem cell according to claim 12, wherein the adipose-derived stem cell is extracted from the inguinal adipose tissues on both sides of an SD rat.

17. The recombinant adipose-derived stem cell according to claim 9, wherein the temperature for the collagenase digestion is 36-38° C.; and the time for the collagenase digestion is 1-1.5 h.

18. The recombinant adipose-derived stem cell according to claim 5, wherein after extracting the adipose-derived stem cell, the method further comprises identifying the obtained adipose-derived stem cell.

19. The recombinant adipose-derived stem cell according to claim 5, wherein the infection has a multiplicity of infection value of 300-400.

20. Use of the recombinant adipose-derived stem cell according to claim 1 in preparing a medicament for preventing and treating hemophilia A.
